(19) United States
(12) Patent Application Publication
(10) Pub. No.: US 2008/0096800 A1
(43) Pub. Date: Apr. 24, 2008

(54) RAPID MUCOSAL GEL OR FILM INSULIN COMPOSITIONS

(75) Inventors: Roderike Pohl, Sherman, CT (US); Solomon S. Steiner, Mount Kisco, NY (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Biodel, Inc.

(21) Appl. No.: 11/923,591

(22) Filed: Oct. 24, 2007

Related U.S. Application Data
(63) Continuation-in-part of application No. 11/869,724, filed on Oct. 9, 2007.
Application No. 11/869,724 is a continuation-in-part of application No. 11/077,604, filed on Mar. 11, 2005, now Pat. No. 7,279,457.
(60) Provisional application No. 60/552,637, filed on Mar. 12, 2004.
(60) Provisional application No. 60/609,194, filed on Sep. 9, 2004.

(51) Int. Cl. A61K 38/28; A61P 3/10

(52) U.S. Cl. 514/3

(57) ABSTRACT

Gel, powder, suspension, emulsions or film formulations for systemic delivery of insulin with improved stability and rapid onset of action are described herein. The formulations are preferably absorbed to a mucosal surface, most preferably via buccal or sublingual administration, although rectal, vaginal, nasal or ocular administration is possible. The formulations contain insulin in combination with a chelator and dissolution agent, and optionally additional excipients. In the preferred embodiment, the formulation contains human insulin, a zinc chelator such as EDTA and a dissolution agent such as citric acid. Following administration, these formulations are rapidly absorbed into the blood stream. The formulation is preferably a polymeric gel, powder or film which adheres to the mucosal surface, thereby enhancing uptake of the incorporated drug. In the preferred embodiment, this formulation is administered sublingually, most preferably before a meal or after a meal.

### CROSS-REFERENCE TO RELATED APPLICATIONS

This application is a continuation in part of U.S. Ser. No. 11/869,724 entitled “Rapid Acting Drug Delivery Compositions”, to Roderike Pohl and Solomon S. Steiner filed Oct. 9, 2007, which is a continuation-in-part of U.S. Ser. No. 11/077,604 filed Mar. 11, 2005, which claims priority to U.S. Ser. No. 60/552,637, entitled “Sublingual Drug Delivery Compositions” to Roderike Pohl and Solomon S. Steiner filed Mar. 12, 2004, and U.S. Ser. No. 60/609,194, entitled “Sublingual Drug Delivery Compositions” to Roderike Pohl and Solomon S. Steiner filed Sep. 9, 2004. The disclosures in the applications listed above are herein incorporated in their entirety by reference.

### FIELD THE INVENTION

The invention is in the general field of rapid delivery formulations, especially gel or film formulations for application to mucosal surfaces.

### BACKGROUND OF THE INVENTION

Diabetes Overview

Glucose is a simple sugar used by all the cells of the body to produce energy and support life. Humans need a minimum level of glucose in their blood at all times to stay alive. The primary manner in which the body produces blood glucose is through the digestion of food. When a person is not getting this glucose from food digestion, glucose is produced from stores in the tissue and released by the liver. The body's glucose levels are regulated by insulin. Insulin is a peptide hormone that is naturally secreted by the pancreas. Insulin helps glucose enter the body's cells to provide a vital source of energy.

When a healthy individual begins a meal, the pancreas releases a natural spike of insulin called the first-phase insulin release. In addition to providing sufficient insulin to process the glucose coming into the blood from digestion of the meal, the first-phase insulin release acts as a signal to the liver to stop making glucose while digestion of the meal is taking place. Because the liver is not producing glucose and there is sufficient additional insulin to process the glucose from digestion, the blood glucose levels of healthy individuals remain relatively constant and their blood glucose levels do not become too high.

Diabetes is a disease characterized by abnormally high levels of blood glucose and inadequate levels of insulin. There are two major types of diabetes—Type 1 and Type 2. In Type 1 diabetes, the body produces no insulin. In the early stages of Type 2 diabetes, although the pancreas does produce insulin, either the body does not produce the insulin at the right time or the body's cells ignore the insulin, a condition known as insulin resistance.

Even before any other symptoms are present, one of the first effects of Type 2 diabetes is the loss of the meal-induced first-phase insulin release. In the absence of the first-phase insulin release, the liver will not receive its signal to stop making glucose. As a result, the liver will continue to produce glucose at a time when the body begins to produce new glucose through the digestion of the meal. As a result, the blood glucose level of patients with diabetes goes too high after eating, a condition known as hyperglycemia. Hyperglycemia causes glucose to attach unnaturally to certain proteins in the blood, interfering with the proteins' ability to perform their normal function of maintaining the integrity of the small blood vessels. With hyperglycemia occurring after each meal, the tiny blood vessels eventually break down and leak. The long-term adverse effects of hyperglycemia include blindness, loss of kidney function, nerve damage and loss of sensation and poor circulation in the periphery, potentially requiring amputation of the extremities.

Between two and three hours after a meal, an untreated diabetic's blood glucose becomes so elevated that the pancreas receives a signal to secrete an inordinately large amount of insulin. In a patient with early Type 2 diabetes, the pancreas can still respond and secretes this large amount of insulin. However, this occurs at the time when digestion is almost over and blood glucose levels should begin to fall. This inordinately large amount of insulin has two detrimental effects. First, it puts an undue extreme demand on an already compromised pancreas, which may lead to its more rapid deterioration and eventually render the pancreas unable to produce insulin. Second, too much insulin after digestion leads to weight gain, which may further exacerbate the disease condition.

Current Treatments for Diabetes and their Limitations

Because patients with Type 1 diabetes produce no insulin, the primary treatment for Type 1 diabetes is daily intensive insulin therapy. The treatment of Type 2 diabetes typically starts with management of diet and exercise. Although helpful in the short-run, treatment through diet and exercise alone is not an effective long-term solution for the vast majority of patients with Type 2 diabetes. When diet and exercise are no longer sufficient, treatment commences with various non-insulin oral medications. These oral medications act by increasing the amount of insulin produced by the pancreas, by increasing the sensitivity of insulin-sensitive cells, by reducing the glucose output of the liver or by some combination of these mechanisms. These treatments are limited in their ability to manage the disease effectively and generally have significant side effects, such as weight gain and hypertension. Because of the limitations of non-insulin treatments, many patients with Type 2 diabetes deteriorate over time and eventually require insulin therapy to support their metabolism.

Insulin therapy has been used for more than 80 years to treat diabetes. This therapy usually involves administering several injections of insulin each day. These injections consist of administering a long-acting basal injection one or two times per day and an injection of a last acting insulin at meal-time. Although this treatment regimen is accepted as effective, it has limitations. First, patients generally dislike injecting themselves with insulin due to the inconvenience and pain of needles. As a result, patients tend not to comply adequately with the prescribed treatment regimens and are often improperly medicated.

More importantly, even when properly administered, insulin injections do not replicate the natural time-action profile of insulin. In particular, the natural spike of the first-phase insulin release in a person without diabetes results in blood insulin levels rising within several minutes of the entry into the blood of glucose from a meal. By contrast, injected insulin enters the blood slowly, with peak insulin levels occurring within 80 to 100 minutes following the injection of regular human insulin.

A potential solution is the injection of insulin directly into the vein of diabetic patients immediately before eating a meal. In studies of intravenous injections of insulin, patients exhibited better control of their blood glucose for 3 to 6 hours following the meal. However, for a variety of medical reasons, intravenous injection of insulin before each meal is not a practical therapy.

One of the key improvements in insulin treatments was the introduction in the 1990s of rapid-acting insulin analogs, such as Humalog®, Novolog® and Apidra®. However, even with the rapid-acting insulin analogs, peak insulin levels typically occur within 50 to 70 minutes following the injection. Because the rapid-acting insulin analogs do not adequately mimic the first-phase insulin release, diabetics using insulin therapy continue to have inadequate levels of insulin present at the initiation of a meal and too much insulin present between meals. This lag in insulin delivery can result in hyperglycemia early after meal onset. Furthermore, the excessive insulin between meals may result in an abnormally low level of blood glucose known as hypoglycemia. Hypoglycemia can result in loss of mental acuity, confusion, increased heart rate, hunger, sweating and faintness. At very low glucose levels, hypoglycemia can result in loss of consciousness, coma and even death. According to the American Diabetes Association, or ADA, insulin-using diabetic patients have on average 1.2 serious hypoglycemic events per year, many of which events require hospital emergency room visits by the patients. Because the time-course of insulin delivery to the blood plays such an important role in overall glucose control, there is significant need for insulin an injectable insulin that reaches the blood more rapidly than the rapid acting insulin analogs.

An effective, non-invasive oral or mucosal delivery system for peptides, in general, and insulin, in particular, has not been developed to date, due to several limiting factors. First, tablets or liquids containing peptides, such as insulin, are readily digested in the harsh stomach environment, and thus require extensive protection to survive and be absorbed. Food effects and individual gastrointestinal (GI) transit times confound a dependable temporal or quantitative delivery.

The lack of effective oral delivery means is further complicated in some cases. For example, insulin is most stable in its hexameric form (six insulin monomers assembled around zinc ions). Therefore, it is preferable to store it in this form for greater shelf-life stability. However, this form is too large for rapid absorption though tissue membranes. U.S. Pat. No. 6,676,931 to Dugger, III discloses liquid sprays that deliver an active agent to the mouth for absorption through the oral mucosa. U.S. Pat. No. 6,676,931 notes that the active agent may be insulin lispro, which is a rapidly-acting human insulin analog that contains hexameric insulin. However, such liquid sprays are not very useful for delivering hexameric insulin due to its poor absorption. Additionally, many active agents are not stable in the liquid form and cannot be stored in liquid form.

Buccal administration using sprays of insulin has been attempted with limited bioavailability since hexameric insulin is not readily absorbed and liquids are eventually swallowed. The administered dose is not rapidly absorbed, and has an absorption profile similar to subcutaneous injection. Also, due to its poor bioavailability and variability, a large dose is required for a useful glucose lowering effect. Thus, it is not a cost effective or therapeutic alternative.

Therefore it is an object of the invention to provide mucosal insulin delivery compositions with improved stability and rapid onset of action.

### SUMMARY OF THE INVENTION

Gel, powder, suspension, emulsions or film formulations for systemic delivery of insulin with improved stability and rapid onset of action are described herein. The formulations are preferably absorbed to a mucosal surface, most preferably via buccal or sublingual administration, although rectal, vaginal, nasal or ocular administration is possible. The formulations contain insulin in combination with a chelator and dissolution agent, and optionally additional excipients. In the preferred embodiment, the formulation contains human insulin, a zinc chelator such as EDTA and a dissolution agent such as citric acid. Following administration, these formulations are rapidly absorbed into the blood stream. The formulation is preferably a polymeric gel, powder or film which adheres to the mucosal surface, thereby enhancing uptake of the incorporated drug. In the preferred embodiment, this formulation is administered sublingually, most preferably before a meal or after a meal.

### BRIEF DESCRIPTION OF THE DRAWINGS

FIG. 1 is a three dimensional schematic of insulin showing exposed surface charges and overlaid with molecules (“dissolution and chelating agents”) of appropriate size to mask the charge.

FIG. 2 is a schematic diagram of the transwell device 10 used to measure insulin absorption from a donor chamber 12 through 4-5 layers of immortalized oral epithelial cells 14 on a 0.1 micron filter 16 into a receiver chamber 18.

FIGS. 3a and 3b are graphs comparing in vitro insulin transport (cumulative insulin in microunits) through oral epithelial cells in the transwell system of FIG. 2, with and without 0.45 mg EDTA/ml, as a function of acid selected as dissolution agent. EDTA was constant at 0.45 mg/mL while the acid concentrations were varied as follows. FIG. 3a, Aspartic acid (0.47 mg/mL), Glutamic acid (0.74 mg/mL), Succinic acid (0.41 mg/mL), Adipic acid (0.73 mg/mL) and Citric acid (0.29 mg/mL and 0.56 mg/mL), pH range 3.2-3.8. FIG. 3b, Maleic (0.32 mg/ml), Fumaric acid (1.28 mg/mL) and Oxalic acid (0.32 mg/mL), pH range 2-3. Two time periods (10 and 30 min.) were selected for comparative analysis. Results are mean plus or minus standard error measured, n=4.

FIGS. 4a and 4b are graphs of in vitro insulin transport (cumulative insulin in microunits) through oral epithelial cells in the transwell system shown in FIG. 2, comparing different dissolution agents, with and without 0.56 mg EDTA/mL and acids at the following equimolar (1.50×10⁻³ Mol) concentrations: Aspartic acid (0.20 mg/mL), Glutamic acid (0.22 mg/mL) and citric acid (0.29 mg/ml) (FIG. 4a) and Citric acid at 1.80 mg/mL (FIG. 4b), Two time periods (10 and 30 min.) were selected for comparative analysis.

FIG. 5 is a graph of in vitro insulin transport through oral epithelial cells using the transwell system of FIG. 2 to compare efficacy of different chelators. Transport of insulin (1 mg/mL) from a solution containing glutamic acid, citric acid or HCl to which different chelators at the same molar concentration (4.84×10⁻³ Mol) were added through oral epithelial cells was measured (cumulative insulin, micromoles). The chelators were no chelator (control), EDTA, EGTA, DMSA, CDTA, and TSC.

FIG. 6 is a graph of the release of insulin from an oral formulation over time, expressed as percent released in a period of three minutes.

FIGS. 7 a,b, and c are graphs of the in vivo pharmacokinetic profile of insulin concentration (microunits/mL) over time (minutes) for an oral patch administered sublingually to beagle dogs, wherein the formulation contains insulin in combination with glutamic acid, citric acid, or succinic acid and EDTA.

FIGS. 8 a and b are graphs of the pharmacodynamics and pharmacokinetics of a powder formulation consisting of insulin, EDTA and citric acid administered by sublingual administration to a human patient with Type I diabetics. FIG. 8a is a graph of glucose (mg/dl) over time (minutes); FIG. 8b is a graph of insulin concentration (microunits/mL) over time (minutes).

### DETAILED DESCRIPTION OF THE INVENTION

The insulin formulations of human insulin described here are administered immediately prior to a meal or at the end of a meal. In the preferred embodiment, the formulation combines recombinant human insulin with specific ingredients generally regarded as safe by the FDA. The formulation is designed to be absorbed into the blood faster than the currently marketed rapid-acting insulin analogs when administered to a mucosal surface. One of the key features of the formulation of insulin is that it allows the insulin to disassociate, or separate, from the six molecule, or hexameric, form of insulin to the single molecule, or monomeric, form of insulin and prevents re-association to the hexameric form. It is believed that by favoring the monomeric form, this formulation allows for more rapid delivery of insulin into the blood as the human body requires insulin to be in the form of a single molecule before it can be absorbed into the body to produce its desired biological effects. Most human insulin that is sold for injection is in the hexameric form. This makes it more difficult for the body to absorb, as the insulin hexamer must first disassociate to form dimers and then monomers.

### I. DEFINITIONS

As used herein, “insulin” refers to human or non-human, recombinant, purified or synthetic insulin or insulin analogues, unless otherwise specified.

As used herein, “Human insulin” is the human peptide hormone secreted by the pancreas, whether isolated from a natural source or made by genetically altered microorganisms. As used herein, “non-human insulin” is the same as human insulin but from an animal source such as pig or cow.

As used herein, an insulin analogue is an altered insulin, different from the insulin secreted by the pancreas, but still available to the body for performing the same action as natural insulin. Through genetic engineering of the underlying DNA, the amino acid sequence of insulin can be changed to alter its ADME (absorption, distribution, metabolism, and excretion) characteristics. Examples include insulin lispro, insulin glargine, insulin aspart, insulin glulisine, insulin detemir. THE insulin can also be modified chemically, for example, by acetylation. As used herein, human insulin analogues are altered human insulin which is able to perform the same auction as human insulin.

As used herein, a “Chelator” or “chelating agent”, refers to a chemical compound that has the ability to form one or more bonds to zinc ions. The bonds are typically ionic or coordination bonds. The chelator can be an inorganic or an organic compound. A chelate complex is a complex in which the metal ion is bound to two or more atoms of the chelating agent.

As used herein, a “solubilizing agent”, is a compound that increases the solubility of materials in a solvent, for example, insulin in an aqueous solution. Examples of solubilizing agents include surfactants (TWEENS®); solvent, such as ethanol; micelle forming compounds, such as oxyethylene monostearate; and pH-modifying agents.

As used herein, a “dissolution agent” is an acid that, when added to insulin and EDTA, enhances the transport and absorption of insulin relative to HCl and EDTA at the same pH, as measured using the epithelia cell transwell plate assay described in the examples below. HCl is not a dissolution agent but may be a solubilizing agent. Citric acid a dissolution agent when measured in this assay.

As used herein, an “excipient” is an inactive substance other than a chelator or dissolution agent, used as a carrier for the insulin or used to aid the process by which a product is manufactured. In such cases, the active substance is dissolved or mixed with an excipient.

### II. FORMULATIONS

Formulations include insulin, a chelator and a dissolution agent(s) and, one or more other excipients as required to make a formulation suitable for administration to a mucosal surface, for example, a patch or tablet for sublingual administration. Optional pharmaceutically acceptable excipients include, but are not limited to, diluents, binders, lubricants, antioxidants, buffers, preservatives, disintegrants, colorants, stabilizers, flavors, mucoadhesives and surfactants. In the preferred embodiment, the formulations are suitable for sublingual administration or absorption through mucosal surfaces. Formulations may be prepared in a gel, powder, suspension or film.

The choice of dissolution agent and chelator, the concentration of both the dissolution agent and the chelator, and the pH that the formulation is adjusted to, all have a profound effect on the efficacy of the system. While many combinations have efficacy, the preferred embodiment is chosen for many reasons, including safety, stability, regulatory profile, and performance.

In the preferred embodiment, at least one of the formulation ingredients is selected to mask any charges on the active agent. This may facilitate the transmembrane transport of the insulin and thereby increase both the onset of action and bioavailability for the insulin. The ingredients are also selected to form compositions that dissolve rapidly in aqueous medium. Preferably the insulin is absorbed and transported to the plasma quickly, resulting in a rapid onset of action (preferably beginning within about 5 minutes following administration and peaking at about 15-30 minutes following administration).

The chelator, such as EDTA, chelates the zinc in the insulin, thereby removing the zinc from the insulin solution. This shifts the equilibrium toward the dimeric and monomeric form and retards reassembly into the hexamer state. Since these two forms exist in a concentration-driven equilibrium, as the monomers are absorbed, more monomers are created. Thus, as insulin monomers are absorbed, additional dimers dissemble to form more monomers. The monomeric form has a molecular weight that is less than one-sixth the molecular weight of the hexameric form, thereby markedly increasing both the speed and quantity of insulin absorbed. To the extent that the chelator (such as EDTA) and/or dissolution agent (such as citric acid) hydrogen bond with the insulin, it is believed that it masks the charge on the insulin, facilitating its transmembrane transport and thereby increasing both the onset of action and bioavailability for insulin.

Insulin

The insulin can be recombinant or purified from a natural source. The insulin can be human or non-human. Human is preferred. In the most preferred embodiment, the insulin is human recombinant insulin. Recombinant human insulin is available from a number of sources. The insulin may also be an insulin analogue which may be based on the amino acid sequence of human insulin but having one or more amino acids differences, or a chemically modified insulin or insulin analog.

The dosages of the insulin depends on its bioavailability and the patient to be treated. Insulin is generally included in a dosage range of 12-2000 IU/human dose, thus if the insulin has a bioavailability of 5 to 25%, the actual systemic dose delivered to an individual ranges from 3 to 100 IU. For insulin with only 2.5% bioavailability, an oral dose of 4000 IU will deliver a 100 IU systemically available dose. For insulin with a much greater bioavailability, such as 50% bioavailability, delivery of a 3 IU systemically available dose requires an oral dose of only 6 IU.

Dissolution Agents

Certain acids appear to mask charges on the insulin, enhancing uptake and transport, as shown in FIG. 1. Those acids which are effective as dissolution agents include acetic acid, ascorbic acid, citric acid, glutamic, aspartic, succinic, fumaric, maleic, and adipic, relative to hydrochloric acid, as measured in the transwell assay described in the examples below. For example, if the active agent is insulin, a preferred dissolution agent is citric acid. The hydrochloric acid may be used for pH adjustment, in combination with any of the formulations, but is not a dissolution agent.

The range of dissolution agent corresponds to an effective amount of citric acid in combination with insulin and EDTA of between 9.37×10⁻⁴ M to 9.37×10⁻² M citric acid.

Chelators

In the preferred embodiment, a zinc chelator is mixed with the active agent. The chelator may be ionic or non-ionic. Suitable chelators include ethylenediaminetetraacetic acid (EDTA), ethylene-bis(oxyethylene nitro) tetraacetic acid (EGTA), di-, tri-sodium citrate, chlorella, cilantro, 1,2,-Diaminocyclohexanetetraacetic acid (CDTA), dimercaptosuccinic acid (DMSA). Hydrochloric acid is used in conjunction with TSC to adjust the pH, and in the process gives rise to the formation of citric acid, which is a dissolution agent.

In the preferred embodiment, the chelator is EDTA. For example, when the active agent is insulin, it is known that the chelator captures the zinc from the insulin, thereby favoring the dimeric form of the insulin over the hexameric form and facilitating absorption of the insulin by the tissues surrounding the site of administration (e.g. mucosa, or fatty tissue). In addition, the chelator hydrogen may bond to the active agent, thereby aiding the charge masking of the active agent and facilitating transmembrane transport of the active agent.

The range of chelator corresponds to an effective amount of EDTA in combination with insulin and citric acid of between 2.42×10⁻⁴ M to 9.68×10⁻² M EDTA.

Excipients

Pharmaceutical compositions may be formulated in a conventional manner using one or more physiologically acceptable carriers comprising excipients and auxiliaries which facilitate processing of the active compounds into preparations which can be used pharmaceutically. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

In the preferred embodiment, one or more solubilizing agents are included with the insulin agent to promote rapid dissolution in aqueous media. Suitable solubilizing agents include wetting agents such as polysorbates, glycerin and poloxamers, non-ionic and ionic surfactants, food acids and bases (e.g. sodium bicarbonate), and alcohols, and buffer salts for pH control.

Stabilizers are used to inhibit or retard drug decomposition reactions which include, by way of example, oxidative reactions. A number of stabilizers may be used. Suitable stabilizers include polysaccharides, such as cellulose and cellulose derivatives, and simple alcohols, such as glycerol; bacteriostatic agents such as phenol, m-cresol and methylparaben; isotonic agents, such as sodium chloride, glycerol, and glucose; lecithins, such as example natural lecithins (e.g. egg yolk lecithin or soya bean lecithin) and synthetic or semisynthetic lecithins (e.g. dimyristoylphosphatidylcholine, dipalmitoylphosphatidylcholine or distearoyl-phosphatidylcholine; phosphatidic acids; phosphatidylethanolamines; phosphatidylserines such as distearoyl-phosphatidylserine, dipalmitoylphosphatidylserine and diarachidoylphospahtidylserine; phosphatidylglycerols; phosphatidylinositols; cardiolipins; sphingomyelinls.

Gel or Film Forming Excipients

Gels or films are formed by mixing one or more polymers in solution, which gel or solidify by ionic and/or covalent binding. A second layer may also be utilized, composed of a material, such as ethylcellulose, which acts as a waterproof barrier for the drug in films. Suitable materials for a hydrophilic layer include, but are not limited to, starch, chitosans (and chitosan derivatives), pregelatinized starch, gelatin, sugars (including sucrose, glucose, dextrose, lactose and sorbitol), dextrin, maltodextrin, polyethylene glycol, waxes, natural and synthetic gums such as acacia, guar gum, tragacanth, alginate, sodium alginate, alginic acid, alpha lipoid acid, celluloses, including hydroxypropylmethylcellulose, carboxymethylcellulose sodium, hydroxypropylcellulose, hydroxylethylcellulose, ethylcellulose, methyl cellulose, and veegum, hydrogenated vegetable oil, Type I, magnesium aluminum silicate, and synthetic polymers such as acrylic acid and methacrylic acid copolymers, carbomer, methacrylic acid copolymers, methyl methacrylate copolymers, aminoalkyl methacrylate copolymers, polyacrylic acid/polymethacrylic acid, and polyvinylpyrrolidone. Blending or copolymerization sufficient to provide a certain amount of hydrophilic character can be useful to improve wettability and mucoadhesion of the materials. For example, about 5% to about 20% of monomers may be hydrophilic monomers. Hydrophilic polymers such as hydroxylpropylcellulose (HPC), hydroxpropylmethylcellulose (HPMC), carboxymethylcellulose (CMC), hyaluronic acid and chitosans are commonly used for this purpose. These can also be nonionic polymers such as ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, various phospholipids, sodium N-lauryl-β-iminodipropionate, myristoamphoacetate, lauryl betaine and lauryl sulfobetaine.

Flavorings and Colorings

There are a number of colorings and flavorings that are commercially available. Flavorings include mint, lemon, bubble gum, and other standard flavors. Sweeteners can be added, including non-glucose sweeteners, which are particularly advantageous for administration of insulin. Colorings can be red, blue, green, yellow, orange, or any other standard FDA approved color.

### III. METHODS OF MANUFACTURE AND DEVICE CHARACTERISTICS

The formulation may dissolve or release active in a time period ranging from 1 second to 3 minutes, 3 to 5 minutes, 5 to 12 minutes, or 12 to 30 minutes. The preferred dissolution time is less than 3 minutes. Preferably the insulin is absorbed and transported to the plasma quickly, resulting in a rapid onset of action preferably beginning within about 5 minutes following administration and peaking at about 15-30 minutes following administration).

Pharmaceutical compositions may be formulated in a conventional manner using one or more physiologically acceptable carriers comprising excipients and auxiliaries which facilitate processing of the active compounds into preparations which can be used pharmaceutically. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). Proper formulation is dependent upon the route of administration chosen.

The compounds may be complexed with other agents when they are formulated into the desired dosage form. If water-soluble, such formulated complex then may be formulated in an appropriate buffer, for example, phosphate or citrate buffered saline or other physiologically compatible solutions. Alternatively, if the resulting complex has poor solubility in aqueous solvents, then it may be formulated with a non-ionic surfactant such as TWEEN™, or polyethylene glycol.

In one preferred embodiment, the formulation is a sublingual solid formulation that contains insulin, chelator, and dissolution agent, along with other standard excipients, such as poly(vinyl alcohol), glycerin, carboxymethyl cellulose (CMC), and optionally poly(ethylene glycol) and water. In the preferred embodiment the active agent is insulin and the solubilizing agents are ethylenediamintetraacetic acid (EDTA), and citric acid.

The composition may be in the form of a film. The film is a clear or opaque, flexible, thin material. Typical thicknesses range from 0.01 to 2 mm. The film may have any suitable shape, including round, oval, rectangle, or square. The film may be a monolayer, bilayer or trilayer film. In various embodiments, the film is designed to be suitable for sublingual administration. The monolayer film contains insulin and one or more excipients. The bilayer film contains one or more excipients, such as the dissolution agent and/or a zinc chelator, in a first layer, and the insulin in the second layer. This configuration allows the insulin to be stored separated from the dissolution agent and/or chelator, and/or other excipients, and may increase the stability of the active agent, and optionally increases the shelf life of the composition compared to if the excipients and active agent were contained in a single layer. The trilayer film contains three layers of film. Each of the layers may be different, or two of the layers, such as the middle and bottom layers, may have substantially the same composition with the top layer being a hydrophobic polymer mixture.

In one embodiment, the bottom and top layers surround a core layer containing the insulin. The bottom and top layers may contain one or more excipients, such as the dissolution agent and/or zinc chelator. Perferably the bottom and top layers have the same composition. Alternatively, the bottom and top layers may contain different excipient(s), or different amounts of the same excipient(s). The core layer typically contains the active agent, optionally with one or more excipients.

In the preferred embodiment, the film consists of three components including ethocel in the top layer, chitosan as a second layer and EDTA, glutamic or citric acid, geletin and insulin as a third component on top or imbedded or in a “well” inside the chitosan layer. Each layer may contain additional excipients, such as glycerin, polyvinyl alcohol, carboxymethyl cellulose, and optionally PEG (such as PEG 400 or PEG 1600). In one embodiment, a third layer can be located between the insulin layer and the layer containing the other ingredients to further protect the insulin from degradative ingredients located in the other layer during storage. Suitable materials for the protective layer include carboxymethylcellulose sodium, carnauba wax, cellulose acetate phthalate, cetyl alcohol, confectioner's sugar, ethylcellulose, gelatin, hydroxyethyl cellulose, hydroxypropyl methylcellulose, liquid glucose, maltodextrin, methylcellulose, microcrystalline wax, polymethacrylates, polyvinyl alcohol, shellac, sucrose, talc, titanium dioxide, and zein.

By altering the composition of the excipients, the film can be designed to dissolve or release insulin rapidly (less than 30 seconds) or slowly (up to 15 minutes) in order to achieve the desired absorption profile and subsequent effect. The film may dissolve or release in a time period ranging from 3 to 5 minutes, 5 to 12 minutes, or 12 to 30 minutes or longer. Preferably, the film dissolves or releases drug in a time period ranging from 15 seconds to 3 minutes.

A monolayer film is typically formed by first suspending inactive ingredients and the active agent in water. The suspension is transferred, such as by pouring or pipetting, to a sheet or mold. Then the suspension is dried by lyophilization or other drying technique to remove the water and form a film. Films may be made as one large sheet and cut to a desired size, based on the desired dosage. Base film may be purchased commercially. Alternatively formulations containing a single dose may be manufactured by forming the film using a mold. The bilayer and trilayer films are generally formed in the same manner as the monolayer film with the exception that each layer contains only certain ingredients (e.g. one layer contains the active agent and the other layer contains one or more excipients). Active ingredients may be also contained in a “well” formed inside one of the layers. One side of the film may be coated to prevent diffusion away from the mucosal tissue. For example, the coating may be formed by spraying or floating.

### IV. METHODS OF USING FORMULATIONS

The formulations may be administered in a variety of manners, including buccal administration, nasal administration, sublingual administration, rectal administration, vaginal administration, or ocular administration. Buccal or sublingual are preferred. Following administration, the dosage form either dissolves quickly or releases drug from a matrix, releasing the drug or small particles containing drug. The formulation is designed to be rapidly absorbed and transported to the plasma for systemic delivery, Formulations containing insulin may be administered to a type 1 or type 2 diabetic patient before or during a meal. Sufficiently rapid absorption can shut off the conversion of glycogen to glucose in the liver, thereby preventing hyperglycemia, the main cause of complications from diabetes and the first symptom of type 2 diabetes.

The methods and compositions described above will be further understood with reference to the following non-limiting examples.

### EXAMPLES

### Example 1

### In Vitro Comparison of Uptake and Transport of Insulin Using Epithelial Cell Transwell Assay as a Function of Dissolution Agent

Materials and Methods

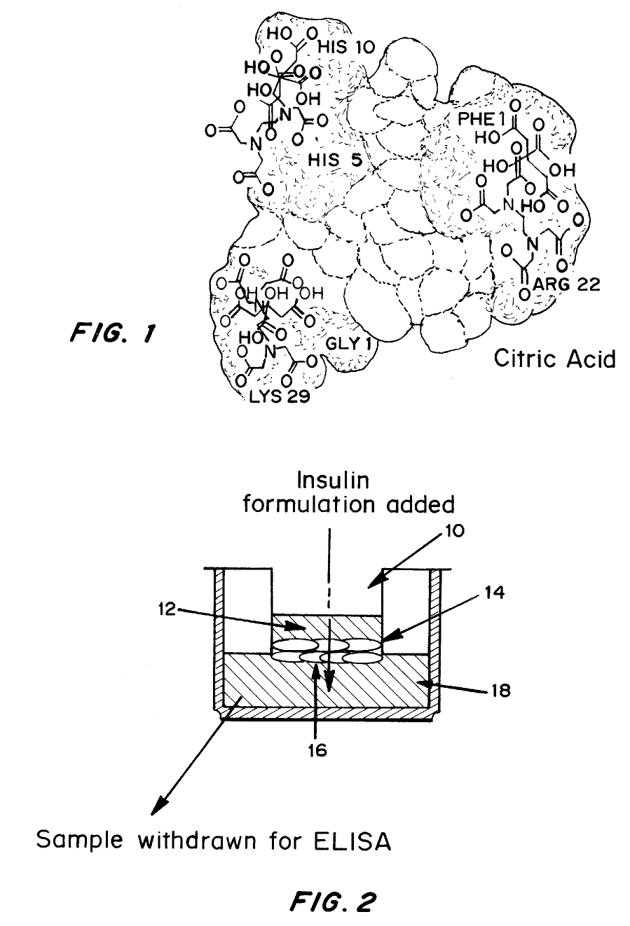

Oral epithelial cells were grown on transwell inserts for two weeks until multiple (4-5 layer) cell layers had formed, as shown in FIG. 2. The transport studies were conducted by adding the appropriate solutions to the donor well and removing samples from the receiver well after 10 minutes. Solutions consisted of water, +/−EDTA (0.45 mg/ml), NaCl (0.85% w/v), 1 mg/ml insulin and a sufficient amount of acid to maintain the pH at 3.8. Insulin amounts in the receiver wells were assayed using ELISA.

Results

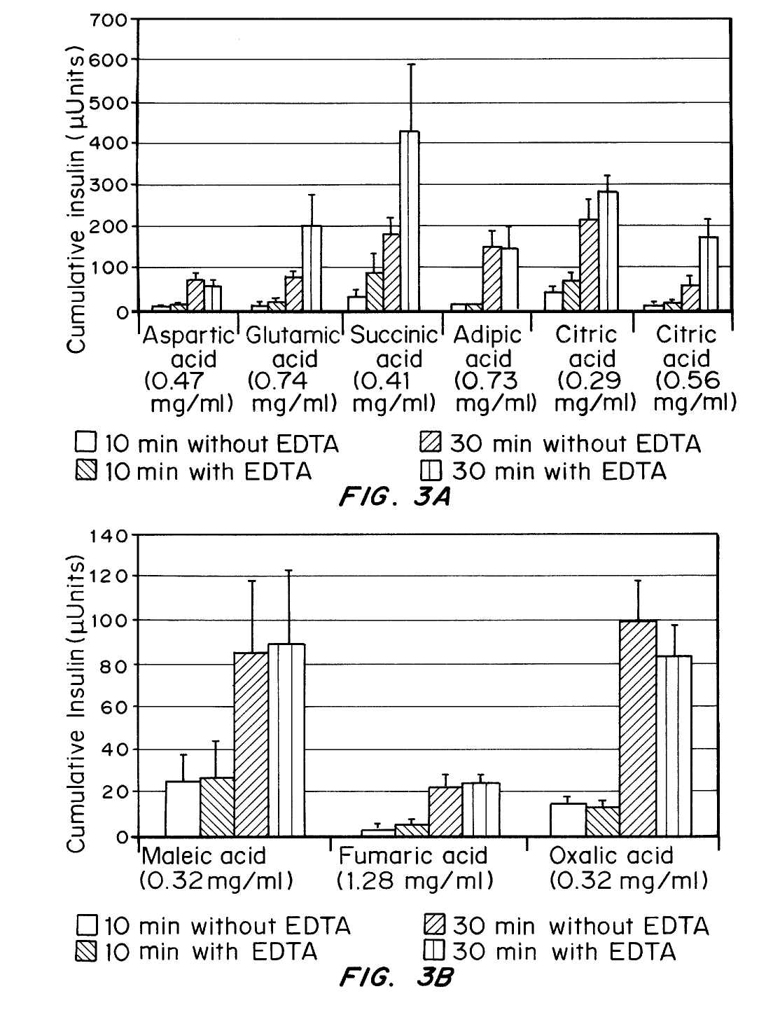

The results shown in FIGS. 3a and 3b demonstrate that some acids are more effective at enhancing uptake and transport of insulin through epithelial cells. These can be readily tested and compared to the results obtained using HCl, thereby providing a standard against which any acid can be tested and determined to be a dissolution agent (i.e., enhancing uptake and transport relative to HCl) or not.

Results obtained with acids with pH range of 3.2-3.8 are grouped in FIG. 3a. Stronger acids (pH<3) are grouped in FIG. 3b.

The results establish that the choice of acid with the same concentration of chelator has a substantial effect on the transport of insulin through cell culture. The preferred acid is citric acid.

### Example 2

### In Vitro Comparison of Uptake and Transport of Insulin Using Epithelial Cell Transwell Assay as a Function of Concentration of Dissolution Agent

Materials and Methods

The materials and methods of Example 1 were used with different concentrations of reagents. In the study, equimolar concentrations of acid and chelator were added. Solutions consisted of water, +/−EDTA (0.56 mg/mL), NaCl (0.85% w/v), 1 mg/mL insulin and an acid: Aspartic acid (0.20 mg/mL), Glutamic acid (0.22 mg/mL) or citric acid (0.20 mg/ml). Citric acid was tested at a higher concentration of 1.8 mg/mL with and without chelator. This data is shown at two time periods, 10 and 30 minutes, post dosing of cell donor chambers.

Results

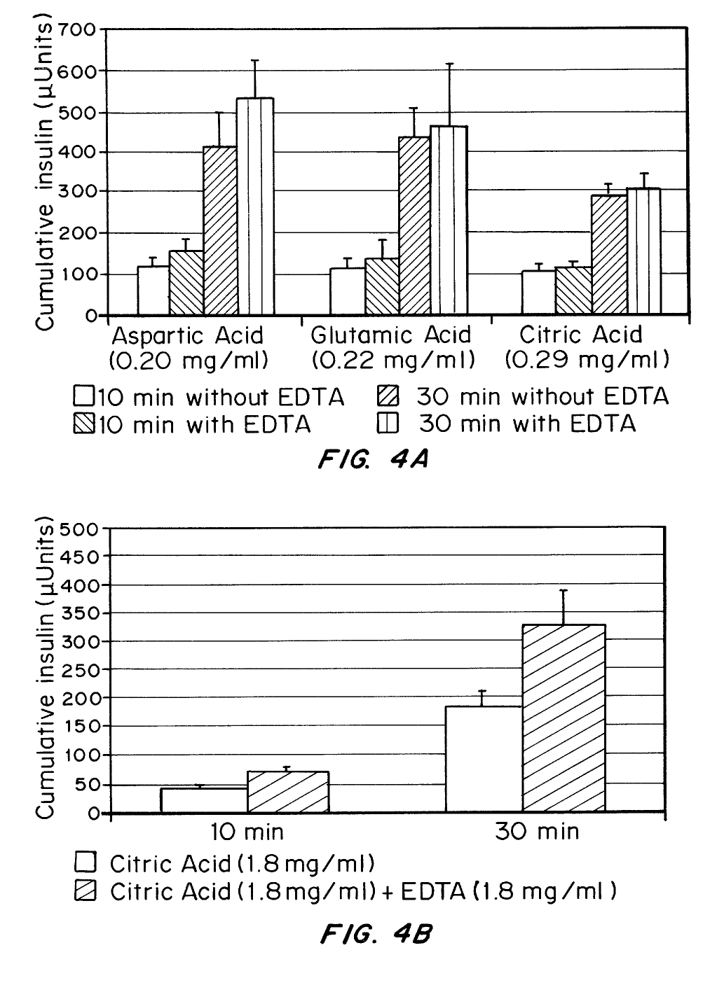

The results obtained with Aspartic acid (0.20 mg/mL), Glutamic acid (0.22 mg/mL) or citric acid (0.29 mg/ml) are shown in FIG. 4a. In this case, there was no significant difference seen with the addition of the chelator.

In contrast, the study using a higher concentration of Citric acid, at 1.80 mg/mL, does show a significant increase (t-test comparison, one sided) upon addition of the chelator to the solution. See FIG. 4b. This demonstrates that concentration of both components is important in optimizing uptake and transport.

### Example 3

### In Vitro Comparison of Uptake and Transport of Insulin Using Epithelial Cell Transwell Assay as a Function of Chelator

Materials and Methods

Oral epithelial cells were grown on transwell inserts for two weeks until multiple (4-5 layer) cell layers had formed. The transport studies were conducted by adding the appropriate solutions to the donor well and removing samples from the receiver well after 10, 20 and 30 minutes.

The solutions were prepared immediately before the transwell experiments in the following way: Citric acid at 1.8 mg/ml was dissolved in 0.85% w/v saline and then one of the following chelators was added to this solution at the concentration shown: EDTA at 1.80 mg/ml, EGTA at 1.84 mg/ml, DMSA at 0.88 mg/ml and TSC at 1.42 mg/ml. Because CDTA was used in its liquid form, citric acid was added directly to the CDTA. In each of these cases, the concentration of chelator was constant at 4.84×10⁻³ moles.

Insulin was then added at 1 mg/ml and the pH was re-adjusted to 3.8 if necessary. A control set of samples using only HCl for pH adjustment are included for comparison. At pH 3.8 alginic acid solidifies, and therefore, was not included for comparison in this example. Transwell experiments were done by adding 0.2 ml of each solution to the donor wells.

Insulin amounts in the receiver wells were assayed using ELISA.

Results

A graph of 30 minute insulin data is shown in FIG. 5. There was significantly more insulin delivered through the cells when citric or glutamic acid was used, except as compared to results obtained with TSC (trisodium citrate). In the case of TSC, HCl was used for pH adjustment. The adjustment of pH generated citric acid, explaining these results.

As demonstrated by these results, enhancement of uptake and transport is dependent on the choice of chelator.

### Example 4

### Release of Insulin from Oral Dosage Forms

Materials and Methods

Oral test formulations were constructed and released into biological media for 3 minutes to determine the amount of insulin that released in 3 min.

The compositions are shown below in Table 1:

| Formu- | | | |
|---|---|---|---|
| lation | | | |
| A | 1^{st} layer | Ethocel + Glycerin | 78.53% |
| | 2^{nd} layer | chitosan | |
| | 3^{rd} layer | Glu + Edta + | |
| | | Glycerin + insulin | |
| | time | 10 min. | |
| B | 1^{st} layer | Ethocel + Glycerin | 82.8%, |
| | 2^{nd} layer | chitosan | 96.36% |
| | 3^{rd} layer | Glu + Edta + | |
| | | insulin(lyophilization) | |
| | time | 3 min., 10 min. | |
| C | 1^{st} layer | Ethocel + Glycerin | 67.03% |
| | 2^{nd} layer | chitosan | |
| | 3^{rd} layer | Glu + Edta + | |
| | | Glycerin + insulin | |
| | | (air dry) | |
| | time | 4 hour | |
| D | 1^{st} layer | Ethocel + Glycerin | 95.90% |
| | 2^{nd} layer | chitosan | |
| | 3^{rd} layer | Glu + Edta + | |
| | | insulin(lyophilization) | |
| | time | 3 min. | |
| E | 1^{st} layer | Ethocel + Glycerin | 46.40% |
| | 2^{nd} layer | chitosan + polyox | |
| | 3^{rd} layer | Glu + Edta + | |
| | | insulin(lyophilization) | |
| | time | 3 min. | |
| F | 1^{st} layer | Ethocel + Glycerin | 26.60% |
| | 2^{nd} layer | chitosan | |
| | 3^{rd} layer | Glu + Edta + | |
| | | insulin + chitosan | |
| | | (lyophilization) | |
| | time | 3 min. | |
| G | 1^{st} layer | Ethocel + Glycerin | 15.80% |
| | 2^{nd} layer | chitosan | |
| | 3^{rd} layer | Glu + Edta + | |
| | | insulin + glycerol | |
| | | (1%)(lyophilization) | |
| | time | 3 min. | |
| H | 1^{st} layer | Ethocel + Glycerin | 24.30% |
| | 2^{nd} layer | chitosan | |
| | 3^{rd} layer | Glu + Edta + | |
| | | insulin + methocel | |
| | | (lyophilization) | |
| | time | 3 min. | |
| I | 1^{st} layer | Ethocel + Glycerin | 17.90% |
| | 2^{nd} layer | chitosan | |
| | 3^{rd} layer | Glu + Edta + | |
| | | insulin + polyox | |
| | | (lyophilization) | |
| | time | 3 min. | |
| J | 1^{st} layer | Ethocel + Glycerin | 50.42% |
| | 2^{nd} layer | chitosan | |
| | 3^{rd} layer | Glu + Edta + | |
| | | insulin + gelatin | |
| | | (0.2%)(lyophilization) | |
| | time | 3 min. | |
| K | 1^{st} layer | Ethocel + Glycerin | 46.68% |
| | 2^{nd} layer | chitosan | |
| | 3^{rd} layer | Glu + Edta + | |
| | | insulin + gelatin | |
| | | (0.1%)(lyophilization) | |
| | time | 3 min. | |
| L | 1^{st} layer | Ethocel + Glycerin | 90.62% |
| | 2^{nd} layer | chitosan | |
| | 3^{rd} layer | Glu + Edta + | |
| | | insulin + gelatin | |
| | | (0.05%)(lyophilization) | |
| | | (coated with gelatin) | |
| | time | 3 min. | |
| M | 1^{st} layer | Ethocel + Glycerin | 89.48% |
| | 2^{nd} layer | chitosan | |
| | 3^{rd} layer | Glu + Edta + | |
| | | insulin + gelatin | |
| | | (0.1%)(lyophilization) | |
| | | (coated with gelatin) | |
| | time | 3 min. | |
| N | 1^{st} layer | Ethocel + Glycerin | 67.25% |
| | 2^{nd} layer | chitosan | |
| | 3^{rd} layer | Glu + Edta + | |
| | | insulin + chitosan | |
| | | (0.01%)(lyophilization) | |
| | time | 3 min. | |
| O | 1^{st} layer | Ethocel + Glycerin | 65.47% |
| | 2^{nd} layer | chitosan | |
| | 3^{rd} layer | Glu + Edta + | |
| | | insulin + gelatin | |
| | | (0.005%)(lyophilization) | |
| | time | 3 min. | |
| P | 1^{st} layer | Ethocel + Glycerin | 90% |
| | 2^{nd} layer | chitosan | |
| | 3^{rd} layer | Glu + Edta + | |
| | | insulin(lyophilization) | |
| | | (coated with gelatin) | |
| | time | 3 min. | |
| R | 1^{st} layer | Ethocel + Glycerin | 79.10% |
| | 2^{nd} layer | chitosan | |
| | 3^{rd} layer | Glu + Edta + | |
| | | insulin + gelatin | |
| | | (0.05%)(lyophilization) | |
| | | (coated with gelatin) | |
| | time | 3 min. | |
| | | (Compressed and made | |
| | | flat film) | |
| S | 1^{st} layer | Ethocel + Glycerin | 82.40% |
| | 2^{nd} layer | chitosan | |
| | 3^{rd} layer | Glu + Edta + | |
| | | insulin + gelatin | |
| | | (0.05%)(lyophilization) | |
| | time | 3 min. | |
| | | (Compressed and made | |
| | | flat film) | |
| T | 1^{st} layer | Ethocel + Glycerin | 80.00% |
| | 2^{nd} layer | chitosan | |
| | 3^{rd} layer | Succinic + Edta + | |
| | | insulin + gelatin | |
| | | (0.05%)(lyophilization) | |
| | time | 3 min. | |
| U | 1^{st} layer | Ethocel + Glycerin | 78.30% |
| | 2^{nd} layer | chitosan | |
| | 3^{rd} layer | citric acid + Edta + | |
| | | insulin + gelatin | |
| | | (0.05%)(lyophilization) | |
| | time | 3 min. | |
| V | 1^{st} layer | Ethocel + Glycerin | 85.90% |
| | 2^{nd} layer | chitosan | |
| | 3^{rd} layer | citric acid + Edta + | |
| | | insulin + gelatin | |
| | | (0.05%)(air dry) | |
| | time | 3 min. | |

Release was measured by dropping the formulation into three mls of synthetic saliva (approximately equal to saliva in normal individual), and then measuring the amount of insulin in the synthetic saliva after three minutes, using HPLC, and comparing that value to the total amount of insulin in the formulation.

Results

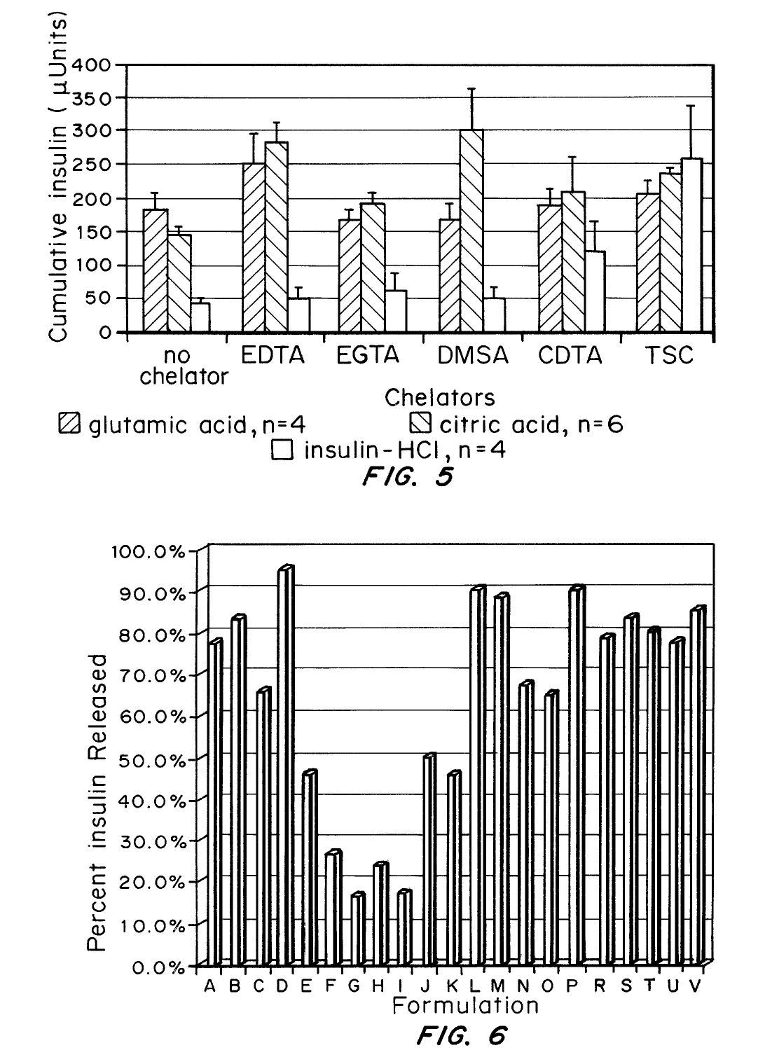

The results are shown in FIG. 6 and demonstrate that one can enhance release by modifying the formulation, and that rapid release was obtained in all cases.

### Example 5

### Preclinical Evaluation in Beagle Dogs

The in vivo pharmacokinetic profile of insulin prepared with glutamic acid, citric acid, or succinic acid in the form of an oral patch administered sublingually to beagle dogs was determined.

Materials and Methods

Six female adult beagle dogs (10-11 kg) were catheterized and given one oral patch on one dosing occasion consisting of either 150 U dose insulin with glutamic, succinic, or citric acid formulations. The oral patch consisted of an ethylcellulose and Chitosan bilayer that was air dried to form a disk. The center of the Chitosan layer was removed and replaced with an insulin formulation, consisting of the 150 U insulin, acid and EDTA. The composition of the ingredients in the insulin formulations were: succinic acid (100 mg succinic acid, 100 mg EDTA, 1.72 mg gelatin), Citric acid (800 mg citric acid, 800 mg EDTA and 1.72 mg gelatin), and Glutamic acid (800 mg glutamic acid, 800 mg EDTA and 1.72 mg gelatin).

Results

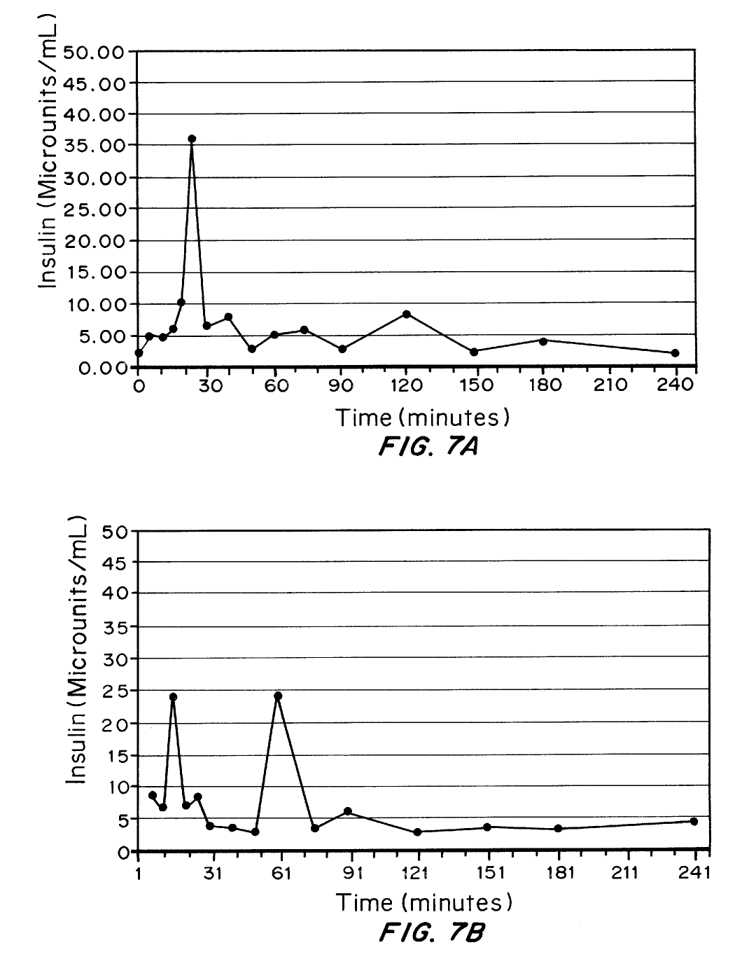

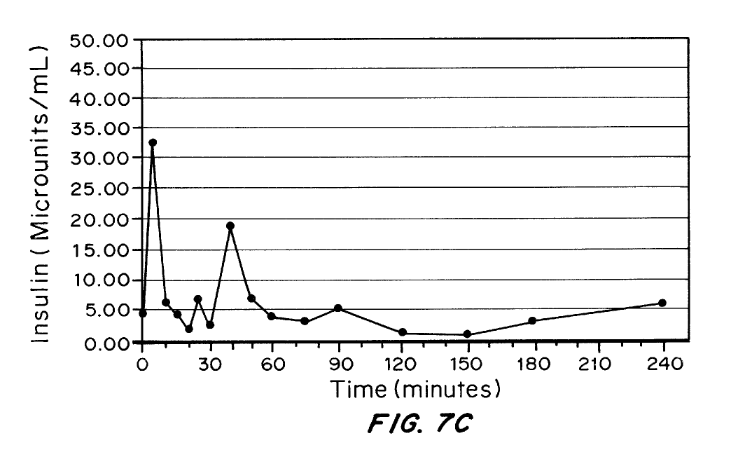

The results are shown in FIGS. 7a, 7b and 7c. In all cases the formulations were effective to rapidly administer insulin to animals via sublingual administration.

### Example 6

### Clinical Evaluation of a Sublingual Administration of a Dry Powder Formulation of Insulin to a Patient with Type 1 Diabetes

Materials and Methods

Powder containing approximately 6 mg insulin, 3 mg EDTA and 3 mg citric acid was prepared for administration by mixing the powders in a test tube. Patient opened his mouth and lifted tongue. Then the entire mixture was sprinkled over the sublingual mucosa of the patient. Patient was instructed to lower the tongue over the powder and not to swallow for as long as possible.

This was administered to one patient. Patient had basal insulin the night prior to administration, and was fasted on the day of the dosing. Powder formulation was evenly poured onto the sublingual surface.

Results

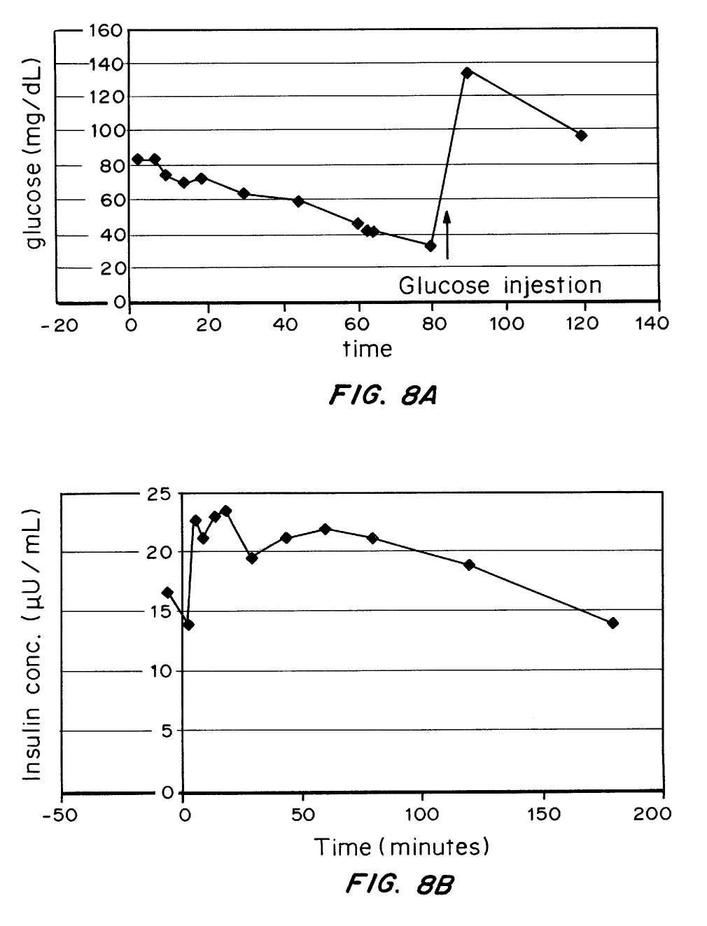

As shown in FIGS. 5a and 8b, plasma insulin levels increased while plasma glucose levels went down following sublingual administration of the insulin powder. Eventually, the blood glucose went down to a point that required ingestion of sugar to prevent hypoglycemia.

Modifications and variations of the methods and materials described herein will be apparent to those skilled in the art and are intended to be encompassed by the following claims.

We claim:

1. A composition for administration of insulin to a mucosal surface comprising insulin and an effective amount of a dissolution agent and a zinc chelator to enhance uptake and transport of the insulin through epithelial cells as compared to insulin alone or insulin in combination with a zinc chelator and HCl in a solid dosage form.

2. The composition of claim 1 wherein the dosage form is selected from the group consisting of a gel, film, powder and patch.

3. The composition of claim 1, wherein the agent is selected from the group consisting of human insulin, animal insulin and insulin analogs.

4. The composition of claim 3, wherein the agent is human insulin.

5. The composition of claim 1, wherein the chelator is selected from the group consisting of ethylenediaminetetraacetic acid (EDTA), EGTA, trisodium citrate (TSC), alginic acid, alpha lipoic acid, dimercaptosuccinic acid (DMSA), CDTA (1,2-diaminocyclohexanetetraacetic acid).

6. The composition of claim 5, wherein the chelator is ethylenediaminetetraacetic acid (EDTA).

7. The composition of claim 1, wherein the dissolution agent is an acid selected from the group consisting of acetic acid, ascorbic acid, citric acid, glutamic, succinic, aspartic, maleic, fumaric, and adipic acid.

8. The composition of claim 7 wherein the dissolution agent is citric acid.

9. The composition of claim 1 wherein the chelator is present in a concentration range corresponding to between 2.42×10⁻⁴ M and 9.68×10⁻²M EDTA.

10. The composition of claim 1 wherein the dissolution agent is present in a concentration range corresponding to between 9.37×10⁻⁴ M and 9.37×10⁻²M citric acid.

11. The composition of claim 1 wherein the zinc chelator is EDTA and the dissolution agent is citric acid and the chelator is present in a concentration of between 2.42×10⁻⁴ M and 9.68×10⁻²M EDTA and the dissolution agent is present in a concentration of between 9.37×10⁻⁴ M and 9.37×10⁻² M citric acid.

12. The composition of claim 1 comprising two or more layers or a coating on a layer, wherein one layer or coating comprises insulin and the other comprises at least one of the dissolution agent or chelator.

13. The composition of claim 1 comprises a coating or backing on one side of the formulation to prevent diffusion of the insulin, wherein the other side is suitable for application to the mucosal surface and release of the insulin.

14. The composition of claim 1 in the form of a film.

15. A method of treating a diabetic individual comprising administering to a mucosal tissue of the individual an effective amount of an insulin composition for administration of insulin to a mucosal surface comprising insulin and an effective amount of a dissolution agent and a zinc chelator to enhance uptake and transport of the insulin through epithelial cells as compared to insulin alone or insulin in combination with a zinc chelator and HCl in the form of a gel, film, powder or patch.

16. The method of claim 15 wherein the composition is applied to the buccal or sublingual area.
